(19) United States
(12) Patent Application Publication
(10) Pub. No.: US 2007/0117206 A1
(43) Pub. Date: May 24, 2007

(54) Methods for clonal derivation of human blastocyst-derived stem cell lines

(76) Inventor: Anders Lindahl, Askim, (SE)

Correspondence Address: BUCHANAN, INGERSOLL & ROONEY PC, POST OFFICE BOX 1404, ALEXANDRIA, VA, 22313-1404, US

(21) Appl. No.: 10/581,901

(22) PCT Filed: Dec. 8, 2004
(86) PCT No.: PCT/EP04/14016

§ 371 Date: Jun. 7, 2006

(30) Foreign Application Priority Data
Dec. 8, 2003 (DK) PA 200301809

(51) Int. Cl. C12N 5/08

(52) U.S. Cl. 435/366

(57) ABSTRACT

Human blastocyst-derived stem cells (hBS) or hBS derived cells, such as, e.g., cells of endothermal, mesodermal, and ectodermal origin, are pluripotent cells with widespread potentials within the areas of, e.g., therapeutic treatment, human developmental biology, and drug discovery processes. The present invention relates to a method for clonal derivation of human blastocyst-derived stem cells (hBS) or hBS derived cells. According to the present invention hBS cell colonies or hBS derived cell colonies are subjected to non-enzymatic treatment for dissociation of the cell colonies to one or more single cells, which are then separately cultivated in a serum based medium and/or serum based conditioned medium to obtain one or more cell clones capable of forming colonies.

### FIELD OF THE INVENTION

The present invention relates to methods and optimized conditions for clonal derivation of a human blastocyst-derived stem cell line (hBS cell line), or a hBS derived cell line. The obtained hBS cell line can be propagated in an undifferentiated state while maintaining its pluripotency both in vitro and in vivo.

### BACKGROUND OF THE INVENTION

Human blastocyst-derived stem cells (hBS) are undifferentiated pluripotent cells to can differentiate to a variety of specialized cells. hBS cell lines have widespread implications for e.g. therapeutic treatment, human developmental biology, and the drug discovery process.

To date, while stem cell lines have been isolated from human blastocysts in various laboratories, only very few of the cell lines have been cloned to obtain a homogenous cell line derived from one single cell, as clonal derivation of hBS stem cell lines are associated with many obstacles such as low survival rate and loss of clones to irreversible differentiation. In previous attempts very low success rates have been reported (<0.5%) referring to number of colonies obtained through number of cells picked. The hBS cells are assumed to be sensitive to even small changes in the micro-environment and are conventionally cultured and passaged as colonies or parts thereof. Accordingly, the dissociation into single cells is one of the critical steps in the art of cloning.

Furthermore, there can be differences between stem cell lines regarding required conditions for clonal expansion, i.e. the conditions that works well with one stem cell line may be ineffective for the cloning and maintenance in an undifferentiated stage of another stem cell line.

As a result there are only few cloned cell lines available. Since the originally described hBS cell lines were not clonally derived from single cells but the inner cell mass of the blastocyst, the pluripotency of the original cell line has not often been demonstrated. Due to this, the formal possibility exists that within a population of homogeneous-appearing cells there are actually multiple precursor or stem cells committed to different lineages. This could mean in fact that no single cell is capable of forming derivatives of all three embryonic germ layers. To determine the pluripotency in human cell cultures, one has to be sure that the cells within a colony are descendants from one single cell. Thus, the obtainment of a pure cell line is essential for the proper use of hBS cells and accordingly, there is a need for methods for obtaining cloned stem cell lines, i.e. stem cell lines derived from one single cell.

The obtainment of a pure population is also of importance in case of hBS derived cell cultures. To rule out the potential of different cells in a mixed population giving rise to different progenies, cloning and subsequent culture and differentiation can verify the potential of e. g. one hBS derived cell of certain origin giving rise to several lineages, such as a hBS derived cell of ectodermal origin giving rise to all three neural lineages; astrocytes, oligodendrocytes, and neurons.

Besides, when culture heterogeneity may occur in a hBS cell line or a hBS derived cell line, such as a mosaically distributed chromosomal aberrations cloning can be used to select and further propagate a cell with desirable characteristics and by doing so obtain culture homogeneity.

The Following Definitions and Abbreviations are Used Herein:

As used herein, the term “blastocyst-derived stem cell” is denoted BS cell, and the human form is termed “hBS cells”.

By the term “feeder cells” or “feeders” are intended to mean cells of one type that are co-cultured with cells of another type, to provide an environment in which the cells of the second type can grow. The feeder cells may optionally be from a different species as the cells they are supporting. The feeder cells may typically be mitotically inactivated when being co-cultured with other cells by irradiation or treatment with an anti-mitotic agent such as mitomycin c, to prevent them from outgrowing the cells they are supporting and specifically the term “mEF” is intended to mean mouse embryonic feeder.

By the terms “feeder-free culture system”, “feeder cell free” or “feeder free” is intended to mean cultures or cell populations wherein less than 10% of the total cells in the culture are feeder cells, such as, e.g., less than 5%, less that 4%, less than 3%, less than 2%, less than 1%, less than 0.5%, less than 0.1% and less than 0.01%. It will be recognized that if a previous culture containing feeder cells is used as a source of hBS cells for the culture to which fresh feeders are not added, there will be some feeder cells that survive the passage. However, after the passage the feeder cells will not proliferate, and only a very small proportion will be viable in continuous cultures.

By the terms “cell clone” or “clone” is intended to mean a cell population derived from a single cell.

By the terms “clonally derived” or “cloned” is intended to mean derived from a single cell.

By the term “substantially single cells” is intended to mean what is believed to be one cell when cells are selected under a microscope, since during the selection can be is difficult to see if more than one cell should be present.

By the term “substantially pure cell population” is intended to mean a cell population derived from a substantially single cell.

By the term “cloning” is intended to mean a method whereby a cell population derived from a single cell is obtained.

By the terms “hBS derived cells” or “hBS cell derivatives” is intended to mean any cells derived from hBS cells in direct line between undifferentiated hBS cells and any fully differentiated cells of endodermal, mesodermal, and/or ectodermal origin. Accordingly, the definition include neural, myocardial, hepatic and pancreatic cells as well as hepatocytes, beta-cells, cardiomyocytes, chondrocytes, osteocytes, keratinocytes, neurons, oligodendrocytes and astrocytes.

By the terms “propagation” or “expansion” is intended to mean culturing cells to obtain more cells.

By the term “pluripotency” is intended to mean cells that have the ability to differentiate into more than one type of tissue.

By the terms “support medium” or “support substrate” is intended to mean surfaces or surface treatments comprising components for stimulating cell adhesion, colony formation or for inhibiting differentiation, such as e.g. extra cellular matrix components.

By the term “bFGF” is intended to mean basic fibroblast growth factor. Other growth factors mentioned are e.g. EGF (epithelial growth factor), HGF (hepatocyte growth factor) and/or FGF4 (fibroblast growth factor 4).

### DESCRIPTION OF THE INVENTION

Accordingly, the present invention provides methods and optimized conditions for clonal derivation of a human blastocyst-derived stem cell line (hBS cell line), or a hBS derived cell line. The obtained hBS cell line can be propagated in an undifferentiated state while maintaining its pluripotency both in vitro and in vivo.

The invention also relates to clonal derivation of hBS derived cells, e.g., cells of endodermal, mesodermal, and/or ectodermal origin.

Accordingly, one aspect of the invention relates to a method for clonal derivation of human blastocyst-derived stem cells (hBS cells) or hBS derived cells, the method comprising the steps of
a) subjecting hBS cell colonies or hBS derived cell colonies to non-enzymatic treatment to dissociate the cell colonies to one or more single cells,
b) selecting/picking of one or more single cells,
c) separately cultivating the one or more single cells in a serum based medium and/or serum based conditioned medium,
d) optionally, changing the medium to a serum free medium to obtain one or more cell clones capable of forming colonies.

In some situations it may be difficult to verify that only one or more single cells are selected in step b), depending on the selection procedure, since e.g. two cells can appear as one cell in the microscope. By repeating the cloning procedure one or more times, the risk of selecting two cells of different clonal origin is minimized. Therefore, one embodiment of the invention relates to a method, wherein the steps a)-d) are preceded by the following steps

a1) subjecting hBS cell colonies or hBS derived cell colonies to non-enzymatic treatment to dissociate the cell colonies to substantially single cells,

b1) selecting/picking of one or more substantially single cells,

c1) separately cultivating the one or more substantially single cells in a serum based medium and/or serum based conditioned medium,

d1) optionally, changing the medium to a serum free medium to obtain a substantially pure cell population.

Dissociation Step a) and a1)

The dissociation of the hBS cell colonies in step a) and/or step a1) in a method according to the invention may be done without the use of enzymatic treatment. Normally, cell colonies are dissociated by use of e.g. trypsin, but the present hBS cell lines form very tight complexes, and it may be very difficult to dissociate the cells completely by the use of enzymatic treatment alone resulting in prolonged exposure to enzyme of cells within the population. Besides, a mild chelator, such as EDTA diluted in Ca/Mg free buffered salt solution interacts with the membranes, dissolving cell-cell interactions by binding remaining divalent cat ions such as calcium and magnesium without over-digesting the cell membranes. Certain enzymes, such as trypsin, works by cutting adhesion proteins which can make it more difficult for cells to re-attach after dissociation.

Accordingly, the invention relates to a method wherein the hBS cell colonies or hBS derived cell colonies in step a) and/or step a1) are dissociated by use of a non-enzymatic method.

The non-enzymatic method may comprise the steps of
i) cutting hBS cell colonies or hBS derived cell colonies to obtain smaller units, having a size preferably between about 200 μm×about 200 μm such as between about 100 μm×about 100 μm to whole colonies,
ii) incubating the smaller units with a medium containing a chelator such as, e.g., EDTA, (diluted in a Ca/Mg-free buffered salt solution)
iii) triturating the smaller units to obtain hBS single cells or hBS derived single cells.

The non-enzymatic method for dissociating the cell colonies may further comprise a step of dispersing the hBS single cells or hBS derived single cells in a suitable medium, such as, e.g. a cell free hBS cell conditioned cloning medium (CC-medium), a hBS derived cell free conditioned medium, serum based medium or a hBS culture medium.

Step b), c), b1) and c1)

Once dissociated, single cells are selected and separately cultivated in a serum based medium and/or serum based conditioned medium to increase adhesion and proliferation of hBS cells or hBS derived cells in single cell population by exposing the dissociated cells to adhesion involved proteins such as fibronectin and vimentin contained in the serum.

Inclusion of Step d) and/or step d1)

In order to minimize the risk of differentiation of hBS cells or hBS derived cells, the medium may be changed from a serum based medium to a serum free medium. Therefore, in one embodiment of the invention, step d) and/or step d1) is included. Inclusion of the medium change of step d) and/or step d1) provides for that the hBS cells or hBS derived cells are cultivated in a serum based medium only until adhesion to the feeder layer or feeder free matrix has occurred, where after the medium is changed to a serum free medium. Step d) and/or step d1) may be performed as follows: 50% (v/v) of the medium used in steps b)-d) and/or steps b1)-d1) in a method as described above may be changed to a hBS cell culture medium comprising 77% v/v KnockOut DMEM, 20% v/v Serum Replacement, 1% v/v Glutamax, 1% v/v NEM, 1% v/v PEST and 4 ng/ml bFGF. The first medium change is performed between 12 and 72 hours after the start of the cultivation of the single hBS cells, such as between 16 and 48 hours, such as 24 hours after the start of the cultivation of the single hBS cells.

Further Cultivation

In one embodiment of the invention, the cell clones obtained in step c) and/or step d) or the substantially pure cell populations obtained in step c1) and/or step d1) are further cultivated by propagation on feeder cells in a suitable culture medium, such as VitroHES™ (Vitrolife AB) and passaged by mechanical dissection and subsequent transfer of pieces of colonies to plates or dishes with fresh feeder cells.

In one embodiment of the invention the cloning procedure can be repeated after propagating an earlier obtained clone for an optional number of passages.

Cell Types to be Employed According to the Present Invention

Clonal derivation of cell lines according to the present invention, can be performed using hBS cell colonies or hBS derived cell colonies as the starting material in step a) and/or step a1). The hBS derived cells can be selected from the group consisting of cells of endodermal, mesodermal, and ectodermal origin, as well as fully differentiated cells, such as, e.g., hepatocytes, beta-cells, cardiomyocytes, chondrocytes, osteocytes, keratinocytes, neurons, oligodendrocytes or astrocytes. Accordingly, and depending on the cell type used as starting material, the cells obtained in any of steps c), c1), b) or b1) are also selected from the group consisting of hBS cells, cells of endodermal origin, cells of mesodermal origin, cells of ectodermal origin, and fully differentiated cells.

The human blastocyst derived stem cells or stem cell line to be cloned may be prepared by methods as described in PCT/EP02/14895 and PCT/2004/005033. The hBS derived cells or cell line to be cloned may be prepared by methods as described in PCT/2004/005034.

Conditioned Serum Based Medium

In order to obtain clones of hBS cells, the cultivation in step c) and/or step c1) above may be performed in a medium that promotes propagation of the one or more hBS cells or hBS derived cells.

The present inventors have developed a medium suitable for clonal derivation of hBS cells or hBS derived cells. However, the medium may also be applicable for clonal derivation and cultivation of other stem cell lines, either used in its present form or with suitable adjustments of the components and/or the amounts of the components.

The medium is denoted cell free hBS cell conditioned cloning medium (CC medium) and comprises a concentrated conditioned medium (CC-base), and, optionally, a suitable cultivation medium.

For the clonal derivation of hBS derived cells a hBS derived cell conditioned medium may be used.

A conditioned medium according to the invention is prepared by culturing a population of cells in a medium, and then harvesting the medium.

In a specific method according to the invention the concentrated conditioned medium may be prepared by the following steps:
1) cultivating hBS cells in a serum based medium, such as FCS (Fetal Calf Serum) or human serum to obtain a conditioned medium,
2) collecting the conditioned medium within suitable time intervals,
3) concentrating the conditioned medium, to obtain the concentrated conditioned medium (CC base).

Step 1) may be performed in the presence of feeder cells, such as, any suitable fibroblasts, e.g. embryonic and/or fetal fibroblasts, or under feeder cell free conditions (see below for a more throughout discussion).

The serum based medium may comprise from about 60% v/v to about 90% v/v KO-DMEM (KnockOut Dulbecco's Modified Eagle Medium), from about 5% v/v to about 30% v/v FCS or human serum, from about 1 mM to about 10 mM glucose and from about 1 ng/ml to about 20 ng/ml bFGF (basic fibroblast growth factor).

The serum based medium may further comprise from about 0.1% v/v to about 5% v/v PEST (penicillin/streptomycin), from about 0.1% v/v to about 5% v/v glutamin or a chemical equivalent, such as Glutamax and/or from about 0.1% v/v to about 5% v/v NEAA (non essential amino acids).

In a specific embodiment of the invention, the serum based medium is a FCS based medium, comprising 15% v/v FCS, 3.5 mM glucose, 1% v/v PEST, 1% v/v Glutamax, 1% v/v NEM and 4 ng/ml bFGF in KO-DMEM.

The medium may be collected in step 2) at least every 12^{th} hour, such as, e.g. at least every 18^{th} hour, at least every 24^{th} hour, at least every 36^{th} hour, at least every 48^{th} hour or at least every 60^{th} hour.

Step 3) may be performed by concentrating the conditioned medium by a factor from about 2 to about 10, such as, e.g. from about 2 to about 9, from about 2 to about 8, from about 3 to about 7, from about 4 to about 6 or from about 4 to about 5 by use of a suitable concentrating column.

In a specific embodiment of the invention, the conditioned medium is concentrated by a factor 4, e.g. from about 50 ml to about 12.5 ml, by use of a Centriprep concentration column WM50 spun at 1500 g, but any other column with a suitable cut-off and any other suitable centrifugation conditions may of course be used.

The cell free hBS cell conditioned cloning medium may comprise at least 5% v/v, such as, e.g., at least 7.5% v/v, at least 10% v/v, at least 12.5% v/v, at least 15% v/v, at least 17.5% v/v, at least 20% v/v, at least 25% v/v, at least 30% v/v, at least 35% v/v, at least 40% v/v, at least 45% v/v, at least 50% v/v, at least 55% v/v, at least 60% v/v, at least 65% v/v, at least 70% v/v, at least 75% v/v, at least 80% v/v, at least 85% v/v, at least 90% v/v, at least 95% v/v or 100% v/v of the concentrated conditioned medium (CC-base) and, optionally, a suitable cultivation medium.

In one aspect of the invention the cultivation medium in the cell free hBS cell conditioned cloning medium (CC medium) is KO-DMEM medium.

The cell free hBS cell conditioned cloning medium may further comprise at least one of the following: glucose, Glutamax, NEAA, PEST and/or a growth factor such as, e.g., bFGF, EGF (epithelial growth factor), HGF (hepatic growth factor) and/or FGF4.

In a specific method according to the invention the cell free hBS cell conditioned cloning medium may comprise 82% KO-DMEM (v/v), 15% v/v concentrated conditioned medium (CC base), 3.5 mM D-glucose, 4 ng/ml bFGF, 1% v/v PEST, 1% v/v Glutamax and 1% v/v NEAA.

Non-Conditioned Serum Based Medium

A medium comprising serum has shown to be favorable for the clonal derivation of hBS cells. The medium may be used in steps b)-d) and/or steps b1)-d1) in the method for clonal derivation as described above, and is denoted Fetal Calf Serum (FCS) based medium and comprises at least 5% v/v FCS, such as, e.g., at least 7.5% v/v FCS, at least 10% v/v FCS, at least 12.5% v/v FCS, at least 15% v/v FCS, at least 17.5% v/v FCS, at least 20% v/v FCS, at least 25% v/v FCS, at least 30% v/v FCS, at least 35% v/v FCS, at least 40% v/v FCS or at least 45% v/v FCS and a suitable cultivation medium.

The cultivation medium may be KO-DMEM medium, and may further comprise a growth factor, such as, e.g. bFGF, EGF, HGF and/or FGF4, and/or glucose.

More specific, the FCS based medium may comprise from about 60% v/v to about 90% v/v KO-DMEM medium, from about 5% v/v to about 30% v/v FCS, from about 1 mM to about 10 mM glucose and from about 1 ng/ml to about 20 ng/ml bFGF, and may further comprise NEM and/or an antibiotic, such as PEST and a glutamine source such as Glutamax.

In a specific method according to the invention the serum based medium may contain 82% KO-DMEM (v/v), 15% v/v FCS, 3.5 mM D-glucose, 4 ng/ml bFGF, 1% v/v PEST, 1% v/v Glutamax and 1% v/v NEAA.

Another suitable medium for use in step c) and/or step c1) a method according to the invention is a human serum based medium comprising human serum and a suitable cultivation medium.

Feeder/Feeder Free Culture Conditions

The cultivation of the one or more single hBS cells or hBS derived cells in step c) or the substantially pure hBS cell population or hBS derived cell population in step c1) in a method as described above may be performed in the presence of feeder cells, i.e. cells of one type that are co-cultured with cells of another type, to provide an environment in which the cells of the second type can proliferate. The feeder cells may typically be inactivated when being co-cultured with other cells by irradiation or treatment with an anti-mitotic agent such as mitomycin c, to prevent them from outgrowing the cells they are supporting.

Examples of suitable feeder cells are fibroblasts, such as, e.g. mouse embryonic fibroblasts, human foreskin fibroblasts, fetal skin fibroblasts, fetal muscle fibroblasts, adult skin fibroblasts and fibroblasts derived from hBS cells.

Cells are traditionally cultured on a layer of feeder cells in order to promote cell survival, proliferation and colony formation. Unfortunately, using feeder cells increases production costs, impairs scale-up, and produces mixed cell populations that require the clones to be separated from feeder cell components. Furthermore, for therapeutic applications it will be of greatest importance that the cells are cultured without xenogenic tissue contact, such as, e.g. feeder cells. To detect reactions specific for the hBS cells or hBS derived cells, the cells need to be cultured in absence of other supporting cells, no matter the origin of such.

Accordingly, the invention also relates to a method wherein the cultivation of one or more hBS cells or hBS derived cells in step step c) and/or step c1) is performed under feeder cell free conditions.

The presence of a suitable medium, such as, e.g. a tissue culture medium, and a support medium, i.e. a growth support substrate or coating, is very important when growing cells under feeder free conditions. When cultivating hBS cells on feeder cells, the feeder cells excrete various substances that promote colony formation and proliferation and inhibit the differentiation of the hBS cells. When growing cells under feeder free conditions such substances have to be supplemented to the growth medium or coated on to the surfaces of the tissue culture wells. In one embodiment of the invention the support substrate comprises a component that promotes colony formation and/or cell division and/or adhesion and/or inhibits differentiation of the hBS single cells or hBS derived single cells, such as, e.g. albumin, gelatine, poly-ornithine, fibronectin, vitronectin, agarose, poly-L-lysine, collagen, and/or extracellular matrix components, such as, e.g. Matrigel® or laminin and/or combinations thereof.

Preparation of CC Medium

The invention also relates to a method for preparing a concentrated conditioned medium (CC base), the method comprising
1) cultivating hBS cells in a serum-based medium, such as, e.g., FCS or human serum,
2) collecting the conditioned medium within suitable time intervals,
3) concentrating the conditioned medium, to obtain the concentrated conditioned medium (CC base).

Furthermore, the invention relates to a hBS cell conditioned cloning medium comprising a concentrated conditioned medium prepared by a method as described herein.

The details and particulars relating to the method aspect apply mutatis mutandis to these aspects of the invention.

Kit

In a specific embodiment the invention relates to a kit for performing the method according to the invention. The kit comprises at least two of the following components in separate compartments: a cell free hBS cell conditioned cloning medium (CC medium), a concentrated conditioned medium (CC base), a hBS derived cell conditioned cloning medium, a serum based cloning medium, hBS cell culture medium, and human or mouse embryonic feeder cells.

In another embodiment the invention relates to a kit for performing the method according to the invention. The kit comprises at least two of the following components in separate compartments: a cell free hBS cell conditioned cloning medium (CC medium), a concentrated conditioned medium (CC base), a hBS derived cell conditioned cloning medium, a serum based cloning medium, a hBS cell culture medium, and a suitable support medium comprising a component that promotes colony formation and/or cell division and/or adhesion and/or inhibits differentiation of the hBS single cells, such as, e.g. albumin, gelatin, poly-ornithine, fibronectin, agarose, poly-L-lysine, collagen, and/or extracellular matrix components, such as, e.g. Matrigel® or laminin and/or combinations thereof.

Other Applications

Other aspects of the invention appear from the appended claims. The details and particulars described above and relating to the method according to the invention apply mutatis mutandis to the other aspects of the invention.

### FIGURE LEGENDS

FIG. 1 shows a colony derived from one single cell of cell line AS034 (later referred to as AS034.2)15 days after being seeded. The picture was taken before transfer from the 96-well-plate.

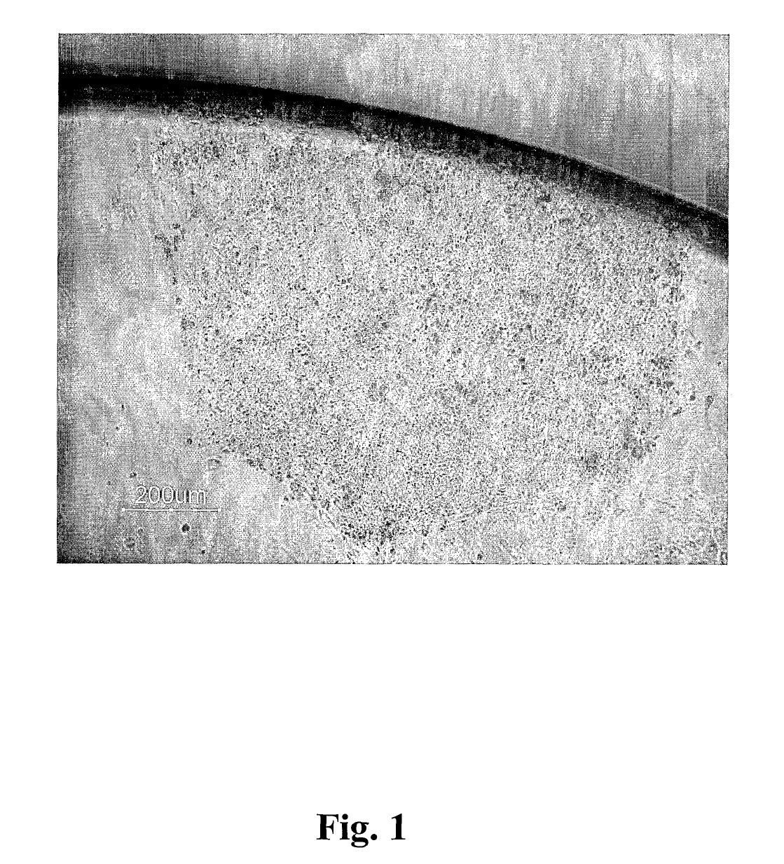

FIG. 2 shows clone AS034.2 after passage 8, 6 days.

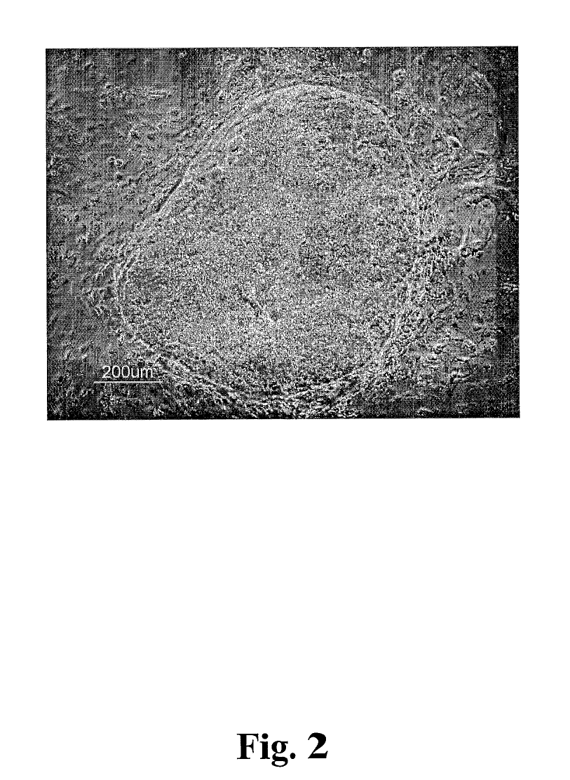

FIG. 3 is a diagram showing the effects of three different media compositions on colony formation from 1000 and 100 cells seeded per well respectively. Five wells in two separate plates were used for each combination of media supplement and cell concentration. The experiment was performed twice and four times respectively for two different cell lines and values presented with SEM (Standard Error of the Mean).

FIG. 4 shows AS034.1—staining positive for the undifferentiated marker SSEA-4.

FIG. 5 shows AS034.1 cultured 12 days and staining positive for the endodermal marker HNF3β (in vitro differentiation).

FIG. 6 shows neuroectodermal tissue from teratoma formation of AS034.1.

FIG. 7 shows secretory epithelium (endoderm) from teratoma formation of AS034.1.

FIG. 8 shows early cartilage (mesoderm) from teratoma formation of AS034.1.

FIG. 9 shows karyotype of parental line SA002 p26 (mosaically trosomy 13)

FIG. 10 shows karyotype of clone SA002.5 p41 (normal karyotype)

The following examples tend to illustrate the invention without limiting it hereto.

### EXAMPLES

### Example 1

Preparation of Concentrated Conditioned Medium (CC Base)

Approximately 10 hBS cell colonies were cultured with mEF cells (150.000 cells were seeded per dish) using a FCS based medium (15% v/v Fetal Calf Serum, 1% v/v PEST, 1% v/v Glutamax, 1% v/v NEAA, 3.5 mM glucose and 4 ng/ml bFGF in KO-DMEM). The medium was collected every second day and concentrated in Centriprep concentration columns WM50 at 1500 g. 50 ml conditioned medium was concentrated 4×(to 12.5 ml) to obtain the concentrated conditioned medium (CC base), aliquoted and frozen. Before use in cloning experiments, the aliquot was diluted 1:50 and sterile filtered.

### Example 2

Standard Culture of hBS Cell Line

ICM outgrowths were passaged to plates with fresh medium and mEF cells by mechanical dissection using a glass capillary as cutting and transfer tool (Swemed Lab International AB, Billdal, Sweden). Established hBS cell lines were routinely passaged every 4-5 days. The hBS cell colonies were mechanically cut into pieces, 200×200 μm, and removed from the culture dish and transferred to a new culture dish with fresh mEF layers cells and hBS cell culture medium.

### Example 3

Preparation of mEF Layer

200 μl MitC (Sigma) were added to 20 ml EMFI medium comprising 500 ml DMEM/Glutamax, 50 ml FCS and 5.5 ml PEST (all Invitrogen). The medium from the mEF cells that were thawed 4 to 5 days earlier was changed to the MitC containing medium and the cells incubated at 37° C. and 5% CO2 for 2.5-3 hours. Preceding plating, mEF cells were washed 3 times in PBS (Invitrogen), dissociated by Trypsin/EDTA solution (Invitrogen) and plated in final concentrations from 100.000 to 150.000 cells in 2 ml medium in IVF-dishes (BD Biosciences) and around 45.000 cells/ml or 9.000 cells/well in 96-well-plates. Prior plating, the wells and dishes were coated with gelatine 2-3 hours (1 g gelatine dissolved in 1 l ddH2O and autoclaved).

### Example 4

Cloning of hBS Stem Cell Line

All the media was pre-warmed before use. Feeder cells were sometimes washed inside the wells twice with cloning medium. The inner part of the colonies was cut and transferred with 300 μm glass capillaries (Swemed Lab International AB) and subsequently incubated with 0.5 mM EDTA in PBS without Ca/Mg for 20 minutes at 37° C. The cells were triturated carefully with a pipette and diluted either in 1) a hBS cell conditioned cloning medium, (KO-DMEM medium supplemented with 15% v/v of concentrated conditioned medium, 3.5 mM glucose, 1% v/v Glutamax (Invitrogen), 1% v/v PEST (Invitrogen), 1% v/v NEAA (invitrogen), and 4 ng/ml bFGF (Sigma)), 2) a KO-DMEM-medium supplemented with 15% v/v FCS, 3.5 mM glucose, 1% v/v Glutamax, 1% v/v PEST, 1% v/v NEAA, and 4 ng/ml bFGF, or 3) a KO-DMEM-medium supplemented with 20% v/v serum replacement (Invitrogen) SR, 1% v/v PEST, 1% v/v Glutamax, 1% v/v NEAA and 4 ng/ml bFGF with or without 3.5 mM glucose. Single cells or substantially single cells were picked with glass capillaries in a stereo microscope and put into individual wells with mEF coated plates. To confirm the colony forming ability of the cells, positive controls were performed (e.g. 100 and 1000 cells and smaller clusters were seeded in individual wells) as well as negative controls (wells without dissociated hBS cells). After 24 hours 50% (v/v) of the medium was changed to fresh hBS culture medium (composition mentioned above) in order to decrease the potential differentiation rate of the colonies. Further medium changes were performed twice a week and the plates were regularly screened for clones.

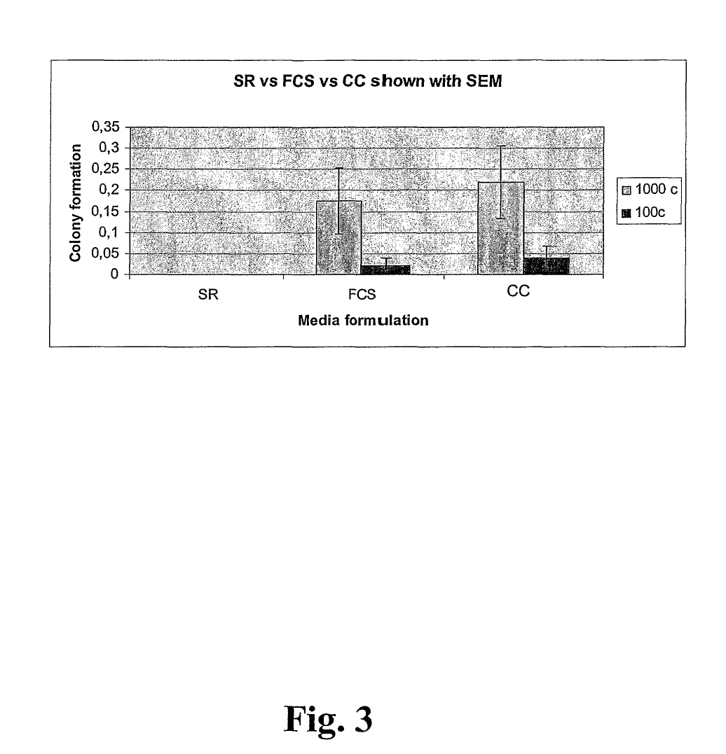

In the inventors efforts to clone cell line AS034, two clones AS034.1 and AS034.2 were obtained, showing a morphology comparable to cell line AS034 from which they were derived. To promote cell survival, cell free hBS cell conditioned medium derived from hBS cells grown in presence of FCS as cloning medium and FCS based medium were used (see above). The overall yield was low; on average from approximately 10³ dissociated single cells one colony resulted. However, both the hBS cell conditioned cloning medium and the FCS based cloning medium gave better results than the use of a serum replacement medium (see FIG. 3).

By definition, clonal derivation of hBS cells is a prerequisite for the strict definition of a pluripotent cell line. The currently available culture conditions for clonal expansion of hBS cells are suboptimal for most hBS cell lines. Unlike mouse BS cells, hBS cells die at high rate when they are dissociated into single cells. For most hBS cell lines used, only 0.1-5% of the plated single cells may be able to generate colonies that could be propagated. Among the few clones that survived the majority are lost due to irreversible differentiation. The present inventors found that concentrated conditioned medium from hBS cells grown in presence of serum, such as, e.g. FCS and optionally, subsequent change to a serum free culture medium after a suitable time interval, promoted cell survival and maintenance of an undifferentiated fate.

In fact one individual cloning experiment of hBS cell line SA002 in FCS based cloning medium resulted in an efficiency of 14% or 20 clones obtained out of 144 single cells picked. A total of 31 clones from SA002 were obtained in the different rounds of experiments. The high yield for this specific hBS cell line is most likely due to this hBS cell line having a higher survival rate of individual cells in suspension as well as a high growth rate of the parental line being inherited by the clones.

Furthermore, culture conditions that may be rate-limiting for maintaining undifferentiated growth of hBS cells, include mEF quality and density, changes in the osmolality, pH, and temperature of the medium, as well as the presence of supplements, such as β-mercaptoethanol.

### Example 5

Cultivation of Clones

Colonies were cut with sharp capillaries and transferred as whole colonies or as clusters to feeder coated IVF dishes or dishes and plates without feeder cells. The following culture and expansion were performed according to example 2 in order to generate sufficient material for characterization and vitrification (Heins et al, PCT/EP02114895, PCT/EP2003/005031).

### Example 6

Repeated Cloning Procedure

The hBS cell line clone AS034.1 was subjected to the same procedure as in example 3-4 resulting in AS034.1.1 after propagation and culture of AS034.1 according to example 2 for 71 passages. The obtained cell line showed identical characteristics as the parental line(s) (see below).

### Example 7

Characterization—Immunohistochemistry

In order to characterize the clones obtained from example 4-5, the cells were fixed in 4% (v/v) PFA (para-formaldehyd) for 15 min at room temperature and exposed to the primary antibodies overnight at 4° C. As secondary antibodies (1:50 FITC- or Cy-3-conjugated antibodies (Southern Biotech) were used.

Primary antibodies against the cell surface antigens SSEA-1, SSEA-3, and SSEA-4 (stage specific embryonic antibodies) as well as the TRA1-60 and TRA1-81 were used to stain undifferentiated hBS cells.

The monoclonal antibodies (mAb) directed against SSEA-1, SSEA-3 and SSEA-4 (Developmental Studies Hybridoma Bank) were used all 1/200. TRA-1-60 and TRA-1-81 (Santa Cruz, Biotechnologies Inc) were used 1/100, neuroectodermal cells were detected with a monoclonal antibody against nestin (BD Transduction Laboratories). Mesodermal cells were detected using a monoclonal antibody against desmin (Chemicon, International) and for endodermal cells the rbAb against HNF3b (Santa Cruz; 1/500) was used. Incubation in primary antiserum was performed at 4° C. overnight. Some cultures were double-stained with DAPI (4′-6′Diamidino-2-phenylindole, Sigma, final concentration 0.1 μg/ml, incubation for 10 minutes).

Alkaline phosphatase activity was tested using a commercially available kit (Sigma-Aldrich) following the manufacturer's instructions.

Results and Discussion

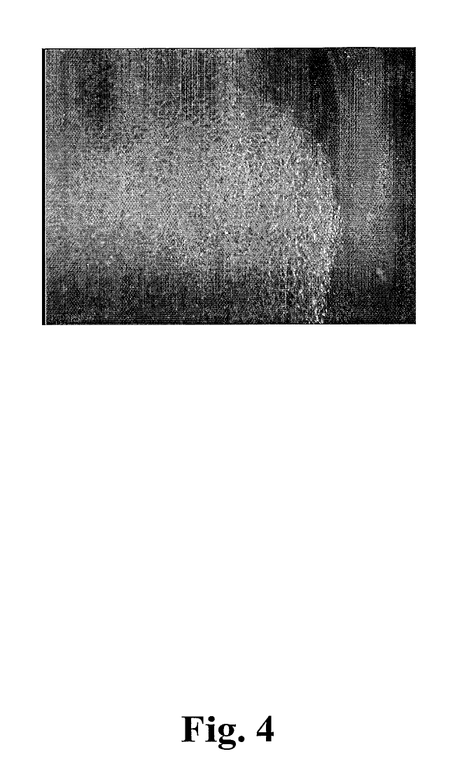

Immunohistochemistry of clone AS034.1, AS034.2, and AS034.1.1 revealed behaviors comparable to the other cell lines in terms of the expression of the cell surface markers SSEA-4, SSEA-3, TRA-1-60 and TRA-1-81. FIG. 4 shows, a colony of AS034.1 positive for the undifferentiated marker SSEA-4. Importantly, SSEA-1 and nestin was not detected in undifferentiated colonies. Furthermore, the clones possessed high levels of alkaline phosphatase (AP) activity, which is normally associated with undifferentiated colonies.

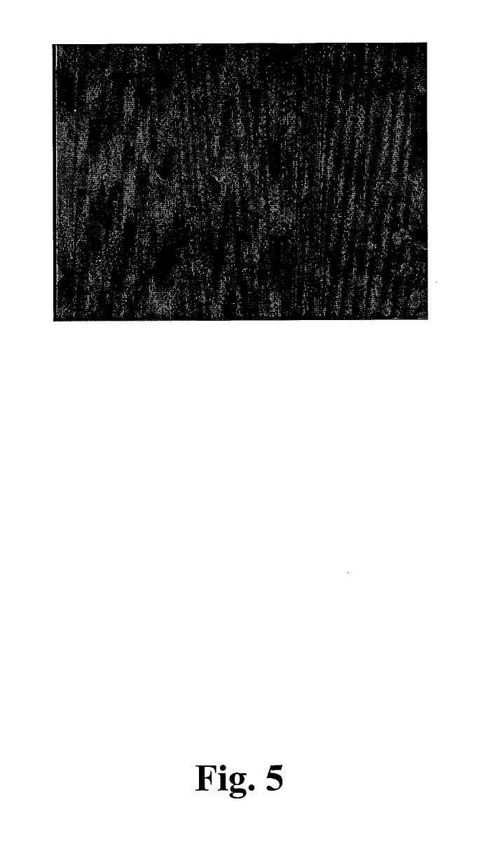

The clones AS034.1 and AS034.2 were further capable of differentiating into ectodermal, mesodermal, and endodermal cell types in vitro. The colonies were kept on mouse feeder layers for more than 7 days without passaging. Already when grown to confluence and allowed to pile up in the culture dish, the clones AS034.1 and AS034.2 differentiated spontaneously, even in the presence of mouse feeder layer cells and human bFGF. Without passing embryoid body stage colonies differentiate spontaneously into a variety of cell types, including all derivatives of the three embryonic germ layers ectoderm, mesoderm and endoderm. Indeed both clones are able to generate in vitro cells of the three germ layers and thus show no differences to cell line AS034 or other previously established hBSC lines (AS034.1 cultured without passaging for 12 days positive for the endoderamal marker HNF3β can be seen in FIG. 5). As mentioned above both AS034.1 and AS034.2 express markers that are essential criteria of undifferentiated human blastocyst-derived stem cell lines.

### Example 8

Characterization—Teratoma Formation in Immunodeficient Mice

Severe combined immunodeficient (SCID)-mice, (Bosma and Carroll, 1991) (C.B-17/IcrCrl-scidBR, Charles River Laboratories, Sulzfeld, Germany) were used as animals host for the xenografted hBS cells. Four to five weeks old animals were anesthetized with intra peritoneal (i.p.) injections of ketamine hydrochloride (Ketalar, Warner Lambert Nordic AB, Solna, Sweden, 75 μg/g mouse) and medetomidine hydrochloride (Domitor, Orion Pharma Corporation, Espoo, Finland, 1 μg/g mouse). hBS cell colonies were mechanically cut into 200×200 μm pieces, washed once in Cryo-PBS (Vitrolife AB) and 20 cell clusters were injected under the kidney capsule using a 300 μm lumen glass transfer pipette (Swemed Lab International AB). The number of cells transferred was approximately 20 000 to 40 000 per organ. Control animals were treated with Cryo-PBS injections and other control animals were grafted with primary brain cells from a littermate. The mice were resuscitated with i.p. injections of atipamezol (Antisedan, Orion Pharma, 1 μg/g mouse), and kept on a heated pad until consciousness. Palpable tumours started to appear three weeks after transplantation. The tumours were allowed to develop for eight weeks before the animals were sacrificed by cervical dislocation. All animals appeared healthy during the eight week period and no animals were deceased due to illness. The tumours were excised and immediately fixated in a 4% (v/v) solution of paraformaldehyde (PFA). The presence of tissues derived from endo-, meso, and ectoderm were confirmed by histological evaluation using hematoxylin-eosin stained paraffin sections.

Results and Discussion

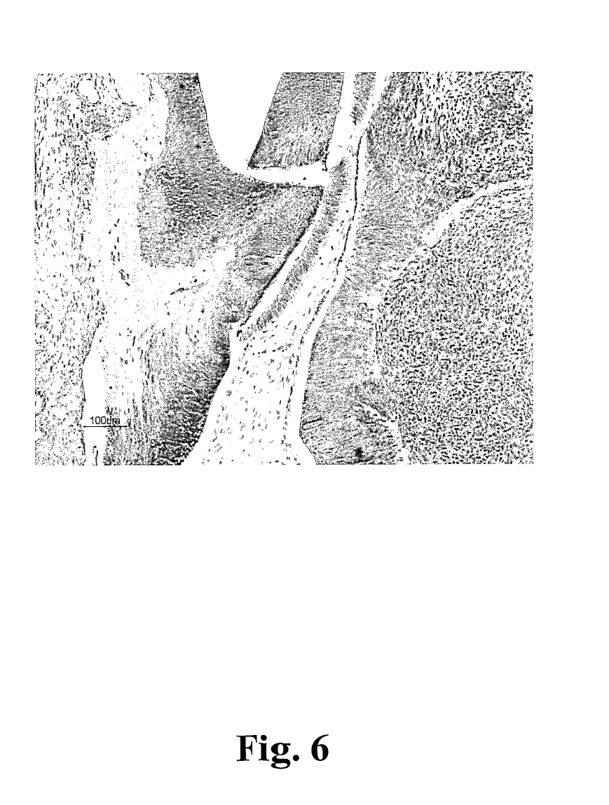

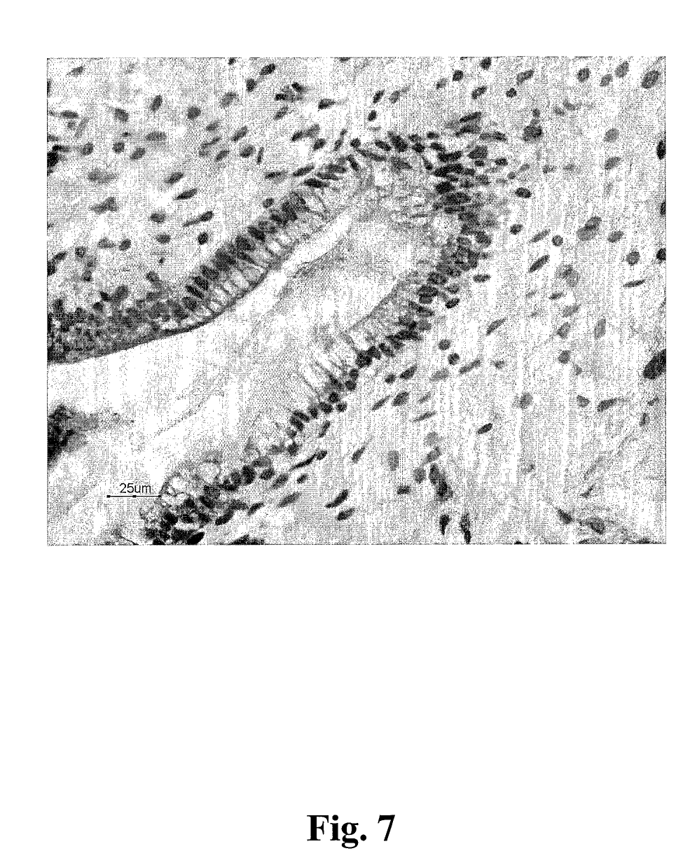

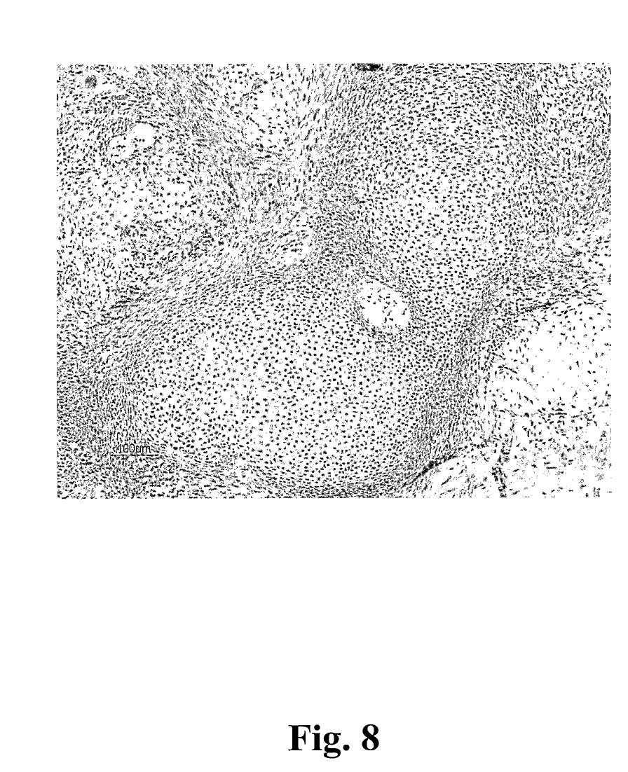

The in vitro and in vivo data suggest the pluripotency of the clones AS034.1 and AS034.2. The teratomas derived from clone AS034.1 and AS034.1.1 showed a similar structure as those of the parental line, AS034. All three germ layers were detected by microscopy analysis of tumor sections, which also suggests the pluripotency of the clone (see FIG. 6-8).

### Example 9

Characterization—FISH Analysis

All clones obtained from SA002 (trisomic chromosome 13) were analysed with fluorescence in situ hybridisation (FISH) analysis, a commercially available kit containing probes for chromosome 13, 18, 21 and the sex chromosomes (X and Y) was used (MultiVysion™ PGT Multicolour Probe Panel; Vysis, Inc., Downers Grove, Ill.) according to the manufacturer's instructions, with minor modifications. For each clone at least 100 nuclei were analyzed. The slides were analyzed in an invert microscope equipped with appropriate filters and software (CytoVision, Applied Imaging, Santa Clara, Calif.).

Results and Discussion

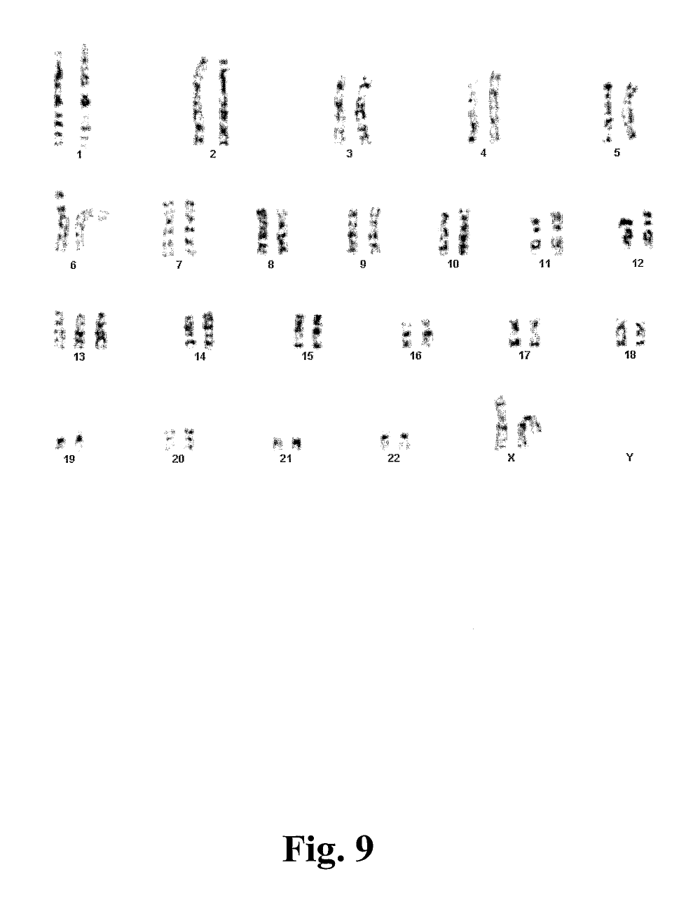

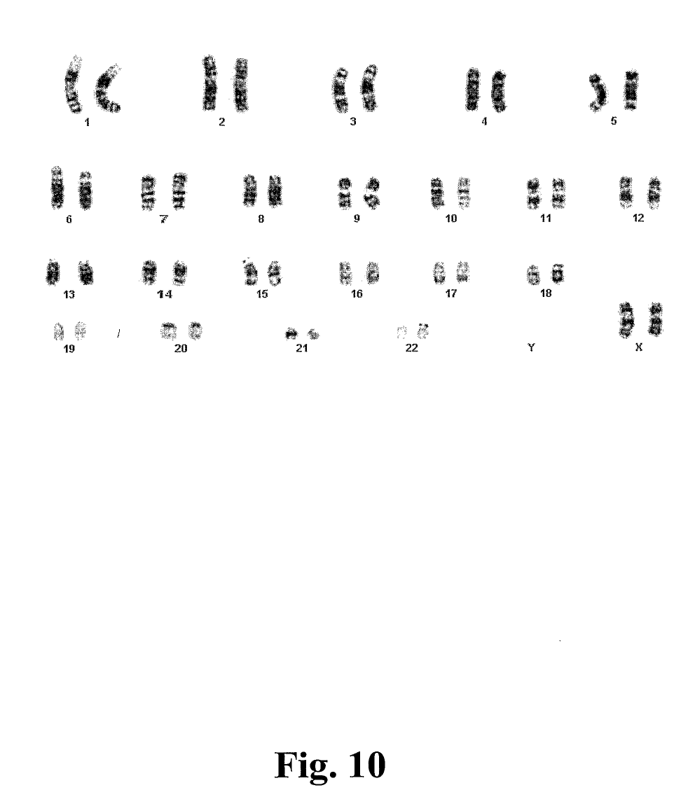

For one of the clones obtained from SA002, namely SA002.5, 94% of the cells showed a normal karyotype with two chromosomes 13 in the first round of FISH analysis. SA002.5 was further expanded, frozen and thawed according to a vitrification technique described elsewhere (PCT/EP2003/005031). FISH analysis have been performed continuously every 5^{th} to 10^{th} passage to monitor potential chromosomal aberrations. Still after 31 passages after cloning the majority of the cells were diploid normal regarding chromosome 13, 18, 21, X and Y. The normal karyotype was confirmed with 10 karyotypes after further 10 passages of culture. This could in fact mean that the cell selected in the cloning experiment and further propagated, was a cell derived from the genetically normal minority of the parental SA002. (See FIGS. 9-10).

### REFERENCES

1. PCT/EP02/14895, “A method for the establishment of a pluripotent human blastocyst-derived stem cell line” in the name of Cellartis AB

2. Thomson, J. A. et al. “Clonally derived human embryonic stem cell lines maintain pluripotency and proliferative potential for prolonged periods of culture”, Developmental Biology 227, 271-278 (2000)

3. Heins N. et al. “Derivation, characterization and differentiation of human embryonic stem cells”, Stem Cells. 2004;22(3):367-76.

4. PCT/2004/005033, “A method for efficient transfer of human blastocyst-derived stem cells from a feeder-supported to a feeder-free culture system”, in the name of Cellartis AB

5. PCT/2004/005034, “A method for the generation of neural progenitor cells”, in the name of Cellartis AB

6. PCT/EP2003/005031, “Cryopreservation of human blastocyst stem cells by use of a closed straw vitrification method”.

7. Yildirim A, Tr., “The Role of Serum on the Adhesion of Cultured Chinese Hamster Lung (CHL) Cells”, J. of Medical Sciences 28 (1998) 383-387

8. Human Embryonic Stem Cells, Kiessling, Ann A, Anderson Scott, World Headquarters, Jones and Bartlett Publishers, Canada, 2003

Acknowledgement

The development of the present invention was financed by the US Government/National Institute of Health, grant number (5R24RR019514-02).

### In Specific Embodiments the Invention Relates to the Following Items

1. A method for clonal derivation of human blastocyst-derived stem cells (hBS) or hBS derived cells, the method comprising the steps of

a) subjecting hBS cell colonies or hBS derived cell colonies to non-enzymatic treatment to dissociate the cell colonies to one or more single cells,

b) selecting/picking of one or more single cells,

c) separately cultivating the one or more single cells in a serum based medium and/or serum based conditioned medium,

d) optionally, changing the medium to a serum free medium to obtain one or more cell clones capable of forming colonies.

2. A method according to item 1, wherein the cultivation in step c) and/or step c1) is performed in a cell free hBS cell conditioned medium (CC medium) or in a cell free hBS derived cell conditioned cloning medium.

3. A method according to item 2, wherein the cell free hBS cell conditioned cloning medium comprises at least 5% v/v, such as, e.g., at least 7.5% v/v, at least 10% v/v, at least 12.5% v/v, at least 15% v/v, at least 17.5% v/v, at least 20% v/v, at least 25% v/v, at least 30% v/v, at least 35% v/v, at least 40% v/v, at least 45% v/v, at least 50% v/v, at least 55% v/v, at least 60% v/v, at least 65% v/v, at least 70% v/v, at least 75% v/v, at least 80% v/v, at least 85% v/v, at least 90% v/v, at least 95% v/v or 100% v/v of a concentrated conditioned medium (CC-base) and, optionally, a suitable cultivation medium.

4. A method according to item 3, wherein the concentrated conditioned medium is prepared by the following steps:
1) cultivating hBS cells in a serum based medium, such as, e.g., FCS (Fetal Calf Serum) based medium or human serum based medium to obtain a conditioned medium,
2) collecting the conditioned medium within suitable time intervals,
3) concentrating the conditioned medium, to obtain the concentrated conditioned medium (CC base).

5. A method according to item 4, wherein step 3) is performed by concentrating the conditioned medium by a factor from about 2 to about 10, such as, e.g. from about 2 to about 9, from about 2 to about 8, from about 3 to about 7, from about 4 to about 6 or from about 4 to about 5 by use of a suitable concentrating column.

6. A method according to items 4 or 5, wherein the serum based medium is FCS based medium and comprises from about 60% v/v to about 90% v/v KO-DMEM medium, from about 5% v/v to about 30% v/v FCS, from about 1 mM to about 10 mM glucose and from about 1 ng/ml to about 20 ng/ml bFGF.

7. A method according to item 6, wherein the FCS based medium further comprises from about 0.1% v/v to about 5% v/v PEST, from about 0.1% v/v to about 5% v/v Glutamax and/or from about 0.1% v/v to about 5% v/v NEAA.

8. A method according to item 7, wherein the FCS based medium comprises 15% v/v FCS, 3.5 mM glucose, 1% v/v PEST, 1% v/v Glutamax, 1% v/v NEM and 4 ng/ml bFGF in KO-DMEM.

9. A method according to any of items 4-8, wherein the medium is collected at least every 12^{th} hour, such as, e.g. at least every 18^{th} hour, at least every 24^{th} hour, at least every 36^{th} hour, at least every 48^{th} hour or at least every 60^{th} hour.

10. A method according to any of items 3-9, wherein the cultivation medium in the cell free hBS cell conditioned cloning medium (CC medium) is KO-DMEM-medium.

11. A method according to item 10, wherein the cell free hBS cell conditioned cloning medium further comprises glucose.

12. A method according to item 10 or 11, wherein the cell free hBS cell conditioned cloning medium (CC medium) further comprises Glutamax, NEM, PEST and/or bFGF.

13. A method according to item 12, wherein the cell free hBS cell conditioned cloning medium comprises 82% v/v KO-DMEM, 15% v/v concentrated conditioned medium (CC base), 3.5 mM D-glucose, 4 ng/ml bFGF, 1% v/v PEST, 1% v/v Glutamax and 1% v/v NEAA.

14. A method according to any of the preceding items, wherein the cultivation in step c) and/or step c1) is performed in a Fetal Calf Serum (FCS) based medium comprising at least 5% v/v FCS, such as, e.g., at least 7.5% v/v FCS, at least 10% v/v FCS, at least 12.5% v/v FCS, at least 15% v/v FCS, at least 17.5% v/v FCS, at least 20% v/v FCS, at least 25% v/v FCS, at least 30% v/v FCS, at least 35% v/v FCS, at least 40% v/v FCS or at least 45% v/v FCS and a suitable cultivation medium.

15. A method according to item 14, wherein the cultivation medium comprises KO-DMEM medium.

16. A method according to item 14 or 15, further comprising a growth factor, such as, e.g. bFGF, EGF, HGF and/or FGF4.

17. A method according to any of items 14-16, further comprising glucose.

18. A method according to item 17, wherein the FCS based medium comprises from about 60% v/v to about 90% v/v KO-DMEM medium, from about 5% v/v to about 30% v/v FCS, from about 1 mM to about 10 mM glucose and from about 1 ng/ml to about 20 ng/ml.

19. A method according to any of items 14-18, wherein the FCS based medium further comprises Glutamax, NEAA and/or PEST.

20. A method according to item 1, wherein the cultivation in step c) and/or step c1) is performed in a serum based medium.

21. A method according to item 1, wherein the cultivation in step c) and/or step c1) is performed in a Human Serum based medium comprising Human Serum and a suitable cultivation medium.

22. A method for preparing a concentrated conditioned medium (CC base), the method comprising
1) cultivating hBS cells in a serum based medium, such as, e.g., FCS (Fetal Calf Serum) based medium or human serum based medium,
2) collecting the conditioned medium within suitable time intervals,
3) concentrating the conditioned medium, to obtain the concentrated conditioned medium (CC base).

23. A method according to item 22, wherein step 3) is performed by concentrating the conditioned medium by a factor from about 2 to about 10, such as, e.g. from about 2 to about 9, from about 2 to about 8, from about 3 to about 7, from about 4 to about 6 or from about 4 to about 5 by use of a suitable concentrating column.

24. A method according to item 22 or 23, wherein the serum based medium is a FCS based medium and comprises from about 60% v/v to about 90% v/v KO-DMEM medium, from about 5% v/v to about 30% v/v FCS, from about 1 mM to about 10 mM glucose and from about 1 ng/ml to about 20 ng/ml bFGF.

25. A method according to item 24, wherein the FCS based medium further comprises from about 0.1% v/v to about 5% v/v PEST, from about 0.1% v/v to about 5% v/v Glutamax and/or from about 0.1% v/v to about 5% v/v NEAA.

26. A method according to item 25, wherein the FCS based medium comprises 15% v/v FCS, 3.5 mM glucose, 1% v/v PEST, 1% v/v NEAA and 4 ng/ml bFGF in KO-DMEM.

27. A method according to any of items 22-26, wherein the medium is collected at least every 12^{th} hour, such as, e.g. at least every 18^{th} hour, at least every 24^{th} hour, at least every 36^{th} hour, at least every 48^{th} hour or at least every 60^{th} hour.

28. A method according to any of items 22-27, wherein the cultivation medium in the cell free hBS cell conditioned cloning medium (CC medium) is KO-DEMEM-medium.

29. A method according to item 28, wherein the cell free hBS cell conditioned cloning medium further comprises glucose.

30. A method according to item 28 or 29, wherein the cell free hBS cell conditioned cloning medium (CC medium) further comprises Glutamax, NEAA, PEST and/or bFGF.

31. A method according to item 30, wherein the cell free hBS cell conditioned cloning medium comprises 81% v/v KO-DMEM, 15% v/v concentrated conditioned medium (CC base), 3.5 mM D-glucose, 4 ng/ml bFGF, 1% v/v PEST, 1% v/v Glutamax and 1% v/v NEAA.

32. A hBS cell conditioned cloning medium comprising a concentrated conditioned medium prepared by a method as described in any of items 22-31.

33. A kit for performing the method described in any of items 1-32, comprising at least two of the following components in separate compartments: a cell free hBS cell conditioned cloning medium (CC medium), a concentrated conditioned medium (CC base), a hBS derived cell conditioned cloning medium, a serum based cloning medium, hBS cell culture medium, and human or mouse embryonic feeder cells.

34. A kit for performing the method described in any of items 1-32, comprising at least two of the following components in separate compartments: cell free hBS cell conditioned cloning medium (CC medium), a concentrated conditioned medium (CC base), a hBS derived cell conditioned cloning medium, a serum based cloning medium, a hBS cell culture medium, and a suitable support medium comprising a component that promotes colony formation and/or cell division and/or adhesion and/or inhibits differentiation of the hBS single cells, such as, e.g. albumin, gelatine, poly-ornithine, fibronectin, agarose, poly-L-lysine, collagen, and/or extracellular matrix components, such as, e.g. Matrigel® or laminin and/or combinations thereof.

1. A method for clonal derivation of human blastocyst-derived stem cells (hBS) or hBS derived cells, the method comprising the steps of
a) subjecting the hBS cell colonies or hBS derived cell colonies to non-enzymatic treatment to dissociate the cell colonies to one or more single cells,
b) selecting/picking of one or more single cells,
c) separately cultivating the one or more single cells in a serum based medium and/or serum based conditioned medium,
d) optionally, changing the medium to a serum free medium to obtain one or more cell clones capable of forming colonies.

2. A method according to claim 1, wherein the steps a)-d) are preceded one or more times by the following steps
a1) subjecting hBS cell colonies or hBS derived cell colonies to non-enzymatic treatment to dissociate the cell colonies to substantially single cells,
b1) selecting/picking of one or more substantially single cells,
c1) separately cultivating the one or more substantially single cells in a serum based medium and/or serum based conditioned medium,
d1) optionally, changing the medium to a serum free medium to obtain a substantially pure cell population.

3. A method according to claim 1, wherein the non-enzymatic method comprises the steps of:
i) cutting hBS cell colonies of hBS derived cell colonies to obtain smaller units,
ii) incubating the smaller units with a medium containing a chelator such as, e.g., EDTA,
iii) triturating the smaller units to obtain hBS single cells or hBS derived single cells.

4. A method according to claim 3, further comprising a step of
iv) dispersing the hBS single cells or hBS derived single cells in a suitable medium, such as, e.g. a cell free hBS cell conditioned cloning medium (CC-medium), a hBS derived cell free conditioned medium, serum based medium or a hBS culture medium.

5. A method according to claim 1, wherein step d) is included.

6. A method according to claim 1, wherein the cell clones obtained in step c) and/or step d) are further cultivated.

7. (canceled)

8. A method according to claim 1, wherein the obtained cell clones are hBS cell clones.

9. A method according to claim 1, wherein the hBS derived cells are selected from the group consisting of cells of endodermal, mesodermal, and ectodermal origin.

10. A method according to claim 1, wherein the obtained cell clones are selected from the group consisting of cells of endodermal, mesodermal, and ectodermal origin.

11. A method according to claim 1, wherein the hBS derived cells are selected from the group consisting of hepatocytes, beta-cells, cardiomyocytes, chondrocytes, osteocytes, keratinocytes, neurons, oligodendrocytes and astrocytes.

12. A method according to claim 1, wherein the obtained cell clones are selected from the group consisting of hepatocytes, beta-cells, cardiomyocytes, chondrocytes, osteocytes, keratinocytes, neurons, oligodendrocytes and astrocytes.

13. A method according to claim 1, wherein the cultivation in step c) is performed in a medium that promotes propagation of the one or more hBS cells or hBS derived cells.

14. A method according to claim 1, wherein step c) is performed in the presence of fibroblasts.

15. A method according to claim 1, wherein step c) is performed under feeder cell free conditions.

16. A method according to claim 15, wherein step c) is performed on a support substrate comprising a component that promotes colony formation and/or cell division and/or adhesion and/or inhibits differentiation of the hBS single cells or hBS derived single cells.

17. (canceled)

18. (canceled)

19. A method according to claim 14, wherein said fibroblasts are selected from the group consisting of mouse embryonic fibroblasts, human foreskin fibroblasts, fetal skin fibroblasts, fetal muscle fibroblasts, adult skin fibroblasts and fibroblasts derived from hBS cells.

20. A method according to claim 16, wherein said component that promotes colony formation and/or cell division and/or adhesion and/or inhibits differentiation of the hBS cells or hBS derived cells is selected from the group consisting of albumin, gelatine, poly-ornithine, fibronectin, vitronectin, agarose, poly-L-lysine, collagen, an extracellular matrix component, and combinations of the foregoing.

21. A method for clonal derivation of human blastocyst-derived stem cells (hBS) or hBS derived cells, the method comprising the steps of
a) subjecting the hBS cell colonies or hBS derived cell colonies to non-enzymatic.
b) selecting/picking of one or more single cells,
c) separately cultivating the one or more single cells in a serum based medium and/or serum based conditioned medium.
d) optionally, changing the medium to a serum free medium to obtain one or more cell clones capable of forming colonies, the steps a)-d) being preceded one or more times by the following steps
a1) subjecting the hBS cell colonies or hBS derived cell colonies to non-enzymatic treatment to disassociate the cell colonies to substantially single cells.
b1) selecting/picking of one or more substantially single cells,
c1) separately cultivating the one or more substantially single cells in a serum based medium and/or serum based conditioned medium,
d1) optionally, changing the medium to a serum free medium to obtain a substantially pure cell population.

22. A method according to claim 21, wherein the non-enzymatic method comprises the steps of
i) cutting hBS cell colonies of hBS derived cell colonies to obtain smaller units,
ii) incubating the smaller units with a medium containing a chelator such as, e.g., EDTA, and
iii) triturating the smaller units to obtain hBS single cells or hBS derived single cells.

23. A method according to claim 22, further comprising a step of iv) dispersing the hBS single cells or hBS derived single cells in a suitable medium, such as, e.g., a cell free hBS cell conditioned cloning medium (CC-medium), a hBS derived cell free conditioned medium, serum based medium or a hBS culture medium.

24. A method according to claim 21, wherein step d) and/or step d1) is included.

25. A method according to claim 21, wherein the cell clones obtained in step c) and/or step d) are further cultivated.

26. A method according to claim 21, wherein the substantially pure cell populations obtained in step c1) and/or step d1) are further cultivated.

27. A method according to claim 21, wherein the obtained cell clones are hBS cell clones.

28. A method according to claim 21, wherein the hBS derived cells are selected from the group consisting of cells of endodermal, mesodermal, and ectodermal origin.

29. A method according to claim 21, wherein the obtained cell clones are selected from the group consisting of cells of endodermal, mesodermal, and ectodermal origin.

30. A method according to claim 21, wherein the hBS derived cells are selected from the group consisting of hepatocytes, beta-cells, cardiomyocytes, chondorocytes, osteocytes, keratinocytes, neurons, oligodendrocytes and astrocytes.

31. A method according to claim 21, wherein the obtained cell clones are selected from the group consisting of hepatocytes, beta-cells, cardiomyocytes, chondrocytes, osteocytes, keratinocytes, neurons, oligodendrocytes and astrocytes.

32. A method according to claim 21, wherein the cultivation in step c) and/or c1) is performed in a medium that promotes propagation of the one or more hBS cells or hBS derived cells.

33. A method according to claim 21, wherein step c) and/or step c1) is performed in the presence of fibroblasts.

34. A method according to claim 21, wherein step c) and/or step c1) is performed under feeder cell free conditions.

35. A method according to claim 34, wherein step c) and/or step c1) is performed on a support substrate comprising a component that promotes colony formation and/or cell division and/or adhesion and/or inhibits differentiation of the hBS single cells or hBS derived single cells.

36. A method according to claim 33, wherein said fibroblasts are selected from the group consisting of mouse embryonic fibroblasts, human foreskin fibroblasts, fetal skin fibroblasts, fetal muscle fibroblasts, adult skin fibroblasts and fibroblasts derived from hBS cells.

37. A method according to claim 35, wherein said component that promotes colony formation and/or cell division and/or adhesion and/or inhibits differentiation of the hBS single cells or hBS derived single cells is selected from the group consisting of albumin, gelatine, poly-ornithine, fibronectin, vitronectin, agarose, poly-L-lysine, collagen, an extracellular matrix component, and combinations of the foregoing.
